(19) United States
(12) Patent Application Publication
(10) Pub. No.: US 2026/0007318 A1
(43) Pub. Date: Jan. 8, 2026

(54) Method and System for Determining a Physiological Parameter

(71) Applicant: Becton, Dickinson and Company, Franklin Lakes, NJ (US)

(72) Inventor: Jeroen Van Goudoever, Amsterdam (NL)

(21) Appl. No.: 18/992,508

(22) PCT Filed: Jul. 8, 2022
(86) PCT No.: PCT/US2022/036524

§ 371 Date: Jan. 8, 2025

(51) Int. Cl. A61B 5/022; A61B 5/00; A61B 5/021

(52) U.S. Cl. CPC A61B 5/02241; A61B 5/02141; A61B 5/7235; A61B 2560/0261

(57) ABSTRACT

The present disclosure relates to a method and corresponding system for a non-invasive determination of a physiological parameter, the method (400) comprising: applying (410) a first pressure cuff (150) to a limb of a living subject, in particular of a human being; applying (420) a second pressure cuff (104) distally to the first pressure cuff (150) to the limb of the subject; inflating (430) the first pressure cuff (150) to a pressure value exceeding a first threshold value; monitoring (440) a pressure signal of the second pressure cuff (104) after the step of inflating the first pressure cuff (150); deriving (450) a measure indicative of mean systemic filling pressure, MSFP, from the monitored pressure signal of the second pressure cuff (104).

### TECHNICAL FIELD

The present disclosure relates to a method and system for determining a physiological parameter. In particular, it relates to a method and system for non-invasive determination of mean systemic filling pressure.

### BACKGROUND OF THE INVENTION

In the context of fluid management of critical care patients, mean systemic filling pressure has recently become of increased interest. Mean systemic filling pressure (MSFP) was defined in 1894 as the pressure in the vascular system during circulatory arrest as published by W. M. Bayliss, E. H. Starling “Observations on venous pressures and their relationship to capillary pressures”. J Physiol. 1894; 16:159-318.

Venous return is determined by the pressure gradient between MSFP and right atrial pressure. MSFP can be measured at the bedside in mechanically ventilated intensive care patients. Using continuous monitoring of cardiac output and central venous pressure, venous return curves can be constructed by performing a series of inspiratory holds. From these venous return curves, the clinician can obtain MSFP, but also other derived parameters as resistance to venous return, systemic compliance, and stressed volume.

A related review article was published by J. J. Maas “Mean systemic filling pressure: its measurement and meaning”, NETH J CRIT CARE-VOLUME 19-NO 1-February 2015, p. 5-11.

Since MSFP represents the pressure in the arterial and venous systems when flow is stopped, the pressure can only be determined during cardiac arrest (and death).

However, there are several alternative methods described to determine MSFP without stopping the heart:
1) inspiratory holds with a mechanical ventilator, using various levels of insufflation pressure, could reveal the relationship between cardiac output and insufflation pressure, and this dependency can be extrapolated to cardiac out equal to zero, the pressure where this occurs is the MSFP. (J. J. Maas, G. F. Geerts, P. C. van den Berg, M. R. Pinsky, J. R. Jansen “Assessment of venous return curve and mean systemic filling pressure in postoperative cardiac surgery patients”. Crit Care Med. 2009; 37:912-8)
2) An arm cuff is used to let the arterial and venous blood equilibrate in the arm, during a phase in which an upper arm cuff is suddenly inflated to above systolic pressure. For that measurement, an invasive arterial blood pressure is needed and an invasive peripheral venous pressure. (J. J. Maas, M. R. Pinsky, B. F. Geerts, R. B. de Wilde and J. R. Jansen (2012). “Estimation of mean systemic filling pressure in postoperative cardiac surgery patients with three methods.” Intensive Care Med 38 (9): 1452-1460)

All previously known methods to determine MSFP rely on invasive pressure measurements.

For the method with the arm cuff inflation, to let the pressure in the arterial and venous system equilibrate in the arm, two invasive transducers are needed. If the arm cuff inflation is kept long enough the arterial and venous pressure both equilibrate to the same level, therefore, in practice only one e.g. arterial blood pressure measurement could be sufficient to be able to obtain the mean systemic filling pressure. The measurement of a slowly decaying or constant arterial blood pressure in an artery cannot be performed with most non-invasive blood pressure measurement devices, since most devices require pulsations. However, these are absent when the proximal cuff is inflated at a high pressure level, e.g. above systolic pressure in the upper arm. Blood pressure measurements based on the volume clamp method follow the full blood pressure waveform in the finger artery whether this intra-arterial blood pressure is constant or pulsating. The combination of finger cuff and volume clamp technology therefore provides a continuous non-invasive measurement of finger arterial blood pressure. This finger arterial blood pressure can be used instead of the arterial pressure, although this pressure may be lower than for instance the radial arterial blood pressure. However, with the proper algorithms a finger blood pressure waveform can be converted to a radial arterial blood pressure waveform.

This why a non-invasive finger arterial blood pressure measurement can be used to obtain the full radial artery waveform non-invasively.

While in many cases, in particular in intensive care environments, an invasive blood pressure measurement is available, this is not always the case. A medical benefit arises from the availability of non-invasively obtained MSFP in cases where no invasive blood pressure measurement for patients is available

It was therefore an object of the present disclosure to provide a method and system which allow a non-invasive determination of MSFP.

### SUMMARY

According to a first aspect a method for a non-invasive determination of a physiological parameter is provided. The method comprises:
applying a first pressure cuff to a limb of a living subject, in particular of a human being,
applying a second pressure cuff distally to the first pressure cuff to the limb of the subject,
inflating the first pressure cuff to a pressure value exceeding a first threshold value,
monitoring a pressure signal of the second pressure cuff after the step of inflating the first pressure cuff,
deriving a measure indicative of mean systemic filling pressure, MSFP, from the monitored pressure signal of the second pressure cuff.

Since the pressure signal of the second pressure cuff is relied upon and since further no invasive operation is employed in the method according to this aspect, a method which allows a non-invasive determination of MSFP is provided.

Preferentially, the second pressure cuff is configured to be operated as a volume clamp blood pressure measurement known in the art. In this preferred embodiment, the change in blood pressure, measured with volume clamp, can be seen from the monitored pressure signal at the second pressure cuff.

According to the present disclosure, the pressure signal at the second pressure cuff is measured or monitored particularly during a phase, in which the blood flow to the second pressure cuff is stopped or at least reduced due to the first pressure cuff inflation to above the first threshold value.

The first pressure cuff is arranged proximally to the second pressure cuff on the same limb of the subject, e.g., on the same arm or leg of the subject in case of a human subject. Thus, inflating the first pressure cuff to a sufficient level, i.e., above the first threshold value, will stop the blood inflow to the distal parts of the limb including the second pressure cuff.

The first threshold value is preferentially predefined or determined during use to be above systolic pressure.

The first pressure cuff is preferentially an arm cuff, the second pressure cuff is preferentially a finger cuff.

In a preferred embodiment the step of monitoring a pressure signal of the second pressure cuff comprises a step of determining a course of a decay of the pressure signal of the second pressure cuff.

After inflating the first pressure cuff to above the first pressure threshold, e.g., to above systolic pressure, the blood pressure in the distal part of the limb, i.e., the location of the second pressure cuff, will decay. This decay in blood pressure will reflect in a decay in the pressure signal of the second pressure cuff. In this preferred embodiment, the course of this decay is determined, for instance approximated as an exponential decay from the obtained values of the pressure signal.

In a preferred embodiment the step of deriving a measure indicative of MSFP comprises the step of deriving an asymptotic value from the determined course of the decay as the measure indicative of MSFP.

The pressure fall in the distal artery, e.g., a finger artery, is due to the equilibrium between blood volume in artery (at a certain pressure) and a venous blood volume at a much lower pressure. The arterial volume equilibrates with the venous volume until the pressure in both compartments is the same, so that a model can be provided to interpret the experienced drop or decay of the pressure signal of the second pressure cuff and the expected asymptotic value. More specifically, due to the resulting blood volume equilibrium, the asymptotic value can be derived as the measure indicative of MSFP.

In a preferred embodiment the step of deriving an asymptotic value comprises the following steps:
fitting the course of the decay of the pressure signal of the second pressure cuff to an exponential decay function, and
determining the asymptotic value from an extrapolation using the exponential decay function.

Experiments and studies showed that the pressure between blood in the artery and venous blood will follow an exponential model and the equilibrium pressure can be expressed as the asymptotic value of this exponential decay function or model.

The parameters of the exponential decay function can be generic parameters or predefined for the specific subject. In further preferred embodiments, the parameters can be adjusted, for instance using machine learning, during execution of the method, i.e., during non-invasively measuring the blood pressure of the subject.

More specifically, in a preferred embodiment, the correlation between the exponential decay function and the decay of the pressure signal can be determined, i.e., the quality of the fitting. In case the correlation does not exceed a predefined threshold, an adaptation of the model parameters to the actual blood pressure values can be performed.

In one preferred example, the asymptotic value is approximately reached after expiration of a certain time period, for instance after more than 30 seconds. In case the pressure signal is monitored for at least the certain time period, e.g., for at least 30 seconds, after inflating the first pressure cuff, the obtained pressure signal can be regarded a true value of the asymptotic value and can be used for adapting the parameters of the exponential decay function.

In a preferred embodiment the method further comprises a step of determining, in particular using the second pressure cuff, a systolic blood pressure of the subject, wherein the first threshold value is set equal to or above the systolic blood pressure.

The first pressure cuff and the second pressure cuff can be used to determine the systolic blood pressure of the subject. It is preferred to obtain the systolic blood pressure at the second pressure cuff since the pressure signal is also monitored at the second pressure cuff to derive the measure indicative of MSFP. If necessary, the systolic blood pressure can also be derived from the cessation of pressure pulsations in the first pressure cuff, indicative of a pressure above systolic pressure.

In a preferred embodiment the step of inflating the first pressure cuff to a pressure value exceeding a first threshold value includes inflating the first pressure cuff using an inflation rate exceeding a second threshold value.

Since the inflation rate exceeds the second threshold, the inflation speed is fast enough to result in a “sudden” inflation and thus a sudden “stop” of blood flow to the second pressure cuff.

If the inflation is not fast enough this can be recognized in the volume clamp measurement with the second cuff as a disturbance in the blood pressure measurement, for instance by a sudden increase in diastolic pressure.

Preferentially, the second threshold value is provided such that the inflation to at least the first threshold value occurs in less than 1 second, further preferentially in less than 0.5 seconds and most preferentially in not more than 0.3 seconds.

In this embodiment the subject preferentially is a human being, and the limb is either of the arms of the human being.

It should be contemplated that an arm cuff can also be applied to a leg of the human being and the finger cuff can also be applied to a toe of the human being. In this embodiment, the limb is a leg of the human being.

In a preferred embodiment the steps of inflating the first pressure cuff, monitoring the pressure signal of the second pressure cuff, and deriving a measure indicative of MSFP are repeated, in particular periodically repeated.

MSFP is changing if the volume in the circulatory system is changing. So, it can be used in strategies to optimize fluid administration in patients or other subjects. So, if changes in blood volume or the compliance of the arteries is changing the MSFP can also change. It is therefore in some embodiments beneficial to reassess MSFP on a recurrent basis.

Since blood pressure and other hemodynamic parameters are continuously measured, characteristic changes in these parameters can be used to trigger an assessment of MSFP.

This is particularly beneficial in the context of the present disclosure since the determination can be performed non-invasively. Thus, repetition of the determination of the measure indicative of MSFP does not impose a substantial burden on the subject.

In a preferred embodiment the steps of inflating the first pressure cuff, monitoring the pressure signal of the second pressure cuff, and deriving a measure indicative of MSFP are periodically repeated at least every hour.

In a further preferred embodiment, a stability of the measure indicative of MSFP can be evaluated, for instance by comparing subsequent measures indicative of MSFP. Depending on the determined stability, the frequency of the periodical repetition can be adapted. In case the measure is determined to be unstable, for instance a change between two subsequent measures exceeds a predefined threshold, in one embodiment the frequency between two measurements is increased.

In a preferred embodiment the influence of the position of the arm relative to heart level can be assessed and compensated using a hydrostatic pressure difference system, e.g. between lower arm and the first pressure cuff or arm cuff (heart level), measuring the position relative to heart level.

In a preferred embodiment changes in the hydrostatic pressure difference system can be assessed and used in a quality index, assessing the possible reduced accuracy due to movement of the arm.

In a preferred embodiment the measurement could work with a first pressure cuff or arm cuff that is not part of the system, but for instance used for auscultatory or oscillatory blood pressure measurement on the arm cuff. Via a e.g. exponential decay of the pressure measured in the second pressure cuff the mean systemic filling pressure can be estimated,

According to a further aspect, a system for a non-invasive determination of a physiological parameter is provided. The system comprises:
a first pressure cuff configured to be applied to a limb of a living subject, in particular of a human being,
a second pressure cuff configured to be applied distally to the first pressure cuff to the limb of the subject,
an inflation control unit configured to inflate the first pressure cuff to a pressure value exceeding a first threshold value,
a pressure signal monitoring unit configured to monitor a pressure signal of the second pressure cuff after the inflation control unit inflated the first pressure cuff,
a deriving unit configured to derive a measure indicative of mean systemic filling pressure, MSFP, from the pressure signal of the second pressure cuff monitored by the pressure signal monitoring unit.

The system according to the present disclosure allows to achieve the same objectives as described in the context of the method according to the present disclosure. Even further, the system can advantageously be configured according to any of the described preferred embodiments of the method according to the present disclosure.

In a preferred embodiment at least one of
a) the first pressure cuff is an arm cuff configured to be applied to an arm of the living subject and
b) the second pressure cuff is a finger cuff configured to be applied to a finger of the arm of the living subject.

In a preferred embodiment at least one of the inflation control unit, the pressure signal monitoring unit and the deriving unit is integrated in a central processing device.

Integrating at least one, preferentially more and most preferred all of the inflation control unit, the pressure signal monitoring unit, and the deriving unit in one central processing device can allow to reduce the complexity of the system. In particular, the control and data processing of the entire system can be performed by the central processing device.

### BRIEF DESCRIPTION OF THE DRAWINGS

The invention will be explained by means of the non-limiting working examples depicted in the following figures. Specifically:

FIG. 1 is a diagram of an environment in which a finger cuff of a blood pressure measurement system and an arm cuff may be implemented,

FIG. 2 is a block diagram illustrating an example environment in which embodiments of the invention may be practiced.

FIG. 3 is a block diagram illustrating example control circuitry.

FIG. 4 is a flow chart exemplarily and schematically illustrating a method according to the present disclosure.

### DETAILED DESCRIPTION

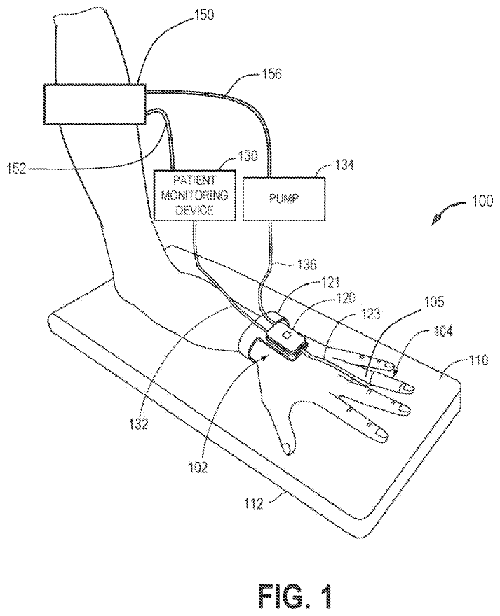

With reference to FIG. 1, an example of a system 100 for determining a physiological parameter and more specifically an environment in which a finger cuff 104 as an example of a second pressure cuff and an arm cuff 150 as an example of a first pressure cuff may be implemented will be described.

As an example, a blood pressure measurement system 102 that includes a finger cuff 104 that may be attached to a patient's finger 105 and a blood pressure measurement controller 120 that may be attached to the patient's body (e.g., a patient's wrist or hand) is shown. The blood pressure measurement system 102 may further be connected to a patient monitoring device 130, and, in some embodiments, a pump 134. Further, finger cuff 104 may include a bladder (not shown) and an LED-PD pair (not shown), which are conventional for finger cuffs. The blood pressure measurement system 102 is particularly preferentially configured to carry out a blood pressure measurement using the volume clamp method.

The system 100 includes in this example a suitable controller, including a processor and memory storing adequate software, to control a pressure of the arm cuff 150 and, using blood pressure measurement system 102, to control the pressure and volume clamp measurement of the finger cuff 104,

In one embodiment, the blood pressure measurement system 102 may include a pressure measurement controller 120 that includes: a small internal pump, a small internal valve, a pressure sensor, and control circuitry. In this embodiment, the control circuitry may be configured to: control the pneumatic pressure applied by the internal pump to the bladder of the finger cuff 104 to replicate the patient's blood pressure based upon measuring the pleth signal received from the LED-PD pair of the finger cuff 104. Further, the control circuitry may be configured to: control the opening of the internal valve to release pneumatic pressure; or the internal valve may simply be an orifice that is not controlled. Additionally, the control circuitry may be configured to: measure the patient's blood pressure by monitoring the pressure of the, bladder based upon the input from a pressure senor, which should be the same as patient's, blood pressure, and may display the patient's blood pressure on the patient monitoring device 130.

In another embodiment, a conventional pressure generating and regulating system may be utilized, in which, a pump 134 is located remotely from the body of the patient. In this embodiment, the blood pressure measurement controller 120 receives pneumatic pressure from remote pump 134 through tube 136 and passes on the pneumatic pressure through tube 123 to the bladder of finger cuff 104. Blood pressure measurement device controller 120 may also control the pneumatic pressure (e.g., utilizing a controllable valve) applied to the finger cuff 104 as well as other functions. In this example, the pneumatic pressure applied by the pump 134 to the bladder of finger cuff 104 to replicate the patient's blood pressure based upon measuring the pleth signal received from the LED-PD pair of the finger cuff 104 and measuring the patient's blood pressure by monitoring the pressure of the bladder may be controlled by the blood pressure measurement controller 120 and/or a remote computing device and/or the pump 134 and/or the patient monitoring device 130. In some embodiments, a blood pressure measurement controller 120 is not used at all and there is simply a connection from the tube 123 to finger cuff 104 from a remote pump 134 including a remote pressure regulatory system, and all processing for the pressure generating and regulatory system, data processing, and display is performed by a remote computing device.

Further, the system 100 comprises an arm cuff 150, which is shown attached to the patient's arm. The arm cuff 150 may be any kind of non-invasive pressure measurement cuff, for instance of the intermittent type known in the art. The arm cuff 150 may be connected to the patient monitoring device 130 through a power/data cable 152. In other embodiments, a separate patient monitoring device is provided for the arm cuff 150, acting as the first pressure cuff according to the present disclosure. Arm cuff 150 may include control electronics and/or a pressure generating and regulatory system or may in other embodiments be connected to remote pump 134 by means of a tube 136. The arm cuff 150 is in the context of the present disclosure capable of being inflated and thus applying a pressure to the arm of the patient, wherein the pressure can exceed a first threshold value such as the systolic blood pressure. Thereby, blood is impeded to flow to the finger cuff 104, i.e., the second pressure cuff according to the present disclosure.

The arm cuff 150 may be any known arm cuff known in the art and may be configured to be manually or automatically inflated. Preferentially, the patient monitoring device 130 or another control device is configured to synchronize the operation of the arm cuff 150 and the finger cuff 104 to improve the non-invasive blood pressure measurement and the accuracy of the determined measure for mean systemic filling pressure.

Continuing with this example, as shown in FIG. 1, a patient's hand may be placed on the face 110 of an arm rest 112 for measuring a patient's blood pressure with the blood pressure measurement system 102. The blood pressure measurement controller 120 of the blood pressure measurement system 102 may be coupled to a bladder of the finger cuff 104 in order to provide pneumatic pressure to the bladder for use in blood pressure measurement. Blood pressure measurement controller 120 may be coupled to the patient monitoring device 130 through a power/data cable 132. Also, in one embodiment, as previously described, in a remote implementation, blood pressure measurement controller 120 may be coupled to a remote pump 134 through tube 136 to receive pneumatic pressure for the bladder of the finger cuff 104. The patient monitoring device 130 may be any type of medical electronic device that may read, collect, process, display, etc., physiological readings/data of a patient including blood pressure, as well as any other suitable physiological patient readings. Accordingly, power/data cable 132 may transmit data to and from patient monitoring device 130 and also may provide power from the patient monitoring device 130 to the blood pressure measurement controller 120 and finger cuff 104. Also, it should be appreciated that a battery may be utilized to provide power to components of the system including the finger cuff 104, the blood pressure measurement controller 120, as well as other system components.

As can be seen in FIG. 1, in one example, the finger cuff 104 may be attached to a patient's finger 105 and the blood pressure measurement controller 120 may be attached on the patient's hand or wrist with an attachment bracelet 121 that wraps around the patient's wrist or hand. The attachment bracelet 121 may be metal, plastic, Velcro, etc. It should be appreciated that this is just one example of attaching a blood pressure measurement controller 120 and that any suitable way of attaching a blood pressure measurement controller to a patient's body or in close proximity to a patient's body may be utilized and that, in some embodiments, a blood pressure measurement controller 120 may not be used at all. The arm cuff 150 is illustrated to be attached to an upper arm of the patient, while in other embodiments the arm cuff 150 can also be attached to any other position of the patient's arm proximal to the finger cuff 104.

It should be appreciated that a heart reference system, which is also known as hydrostatic compensation system, can be used to compensate peripheral blood pressure in the finger cuff 104 for the hydrostatic level shift when the hand is not at heart level. To this end, various solutions are known which are configured for measuring pressure difference between location of the finger cuff 104 and heart level,

It should further be appreciated that the finger cuff 104 may be connected to a blood pressure measurement controller described herein, or a pressure generating and regulating system of any other kind, such as a conventional pressure generating and regulating system that is located remotely from the body of the patient (e.g., a pump 134 located remotely from a patient). The same applies for the arm cuff 150, which can in further alternative embodiments also operate independent from any other component.

Any kind of pressure generating and regulating system that can be used, including but not limited to the blood pressure measurement controller, may be described simply as a pressure generating and regulating system.

As a further example, in some embodiments, there may be no blood pressure measurement controller, at all, and a remote pump 134 that is controlled remotely may be directly connected via a tube 136 and 123 to finger cuff 104 to provide pneumatic pressure to the finger cuff 104 and likewise to arm cuff 150. It Is preferred that at least the finger cuff 104 in system 100 is configured to perform volume clamp measurement in order to reliably provide a measure of intra-arterial blood pressure.

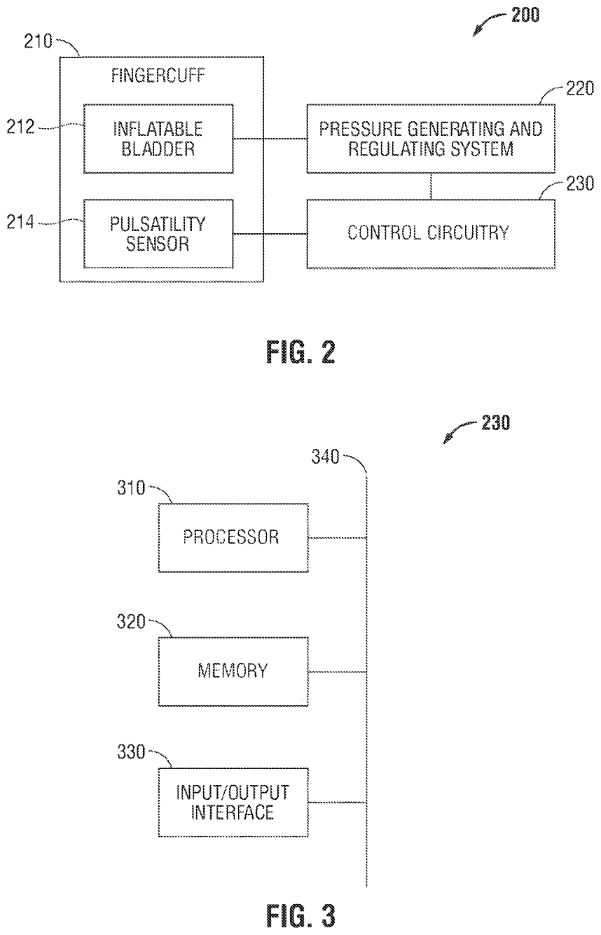

Referring to FIG. 2, a block diagram illustrating an example environment 200 in which embodiments of the disclosure may be practiced is shown. The cuff 210, which may be an example of the finger cuff 104 of FIG. 1 or also of the arm cuff 150 of FIG. 1, may comprise an inflatable bladder 212 and an arterial pulsatility sensor 214. The inflatable bladder 212 may be pneumatically connected to a pressure generating and regulating system 220. The pressure generating and regulating system 220 may generate, measure, and regulate pneumatic pressure that inflates or deflates the bladder 212, and may comprise such elements as a pump, a valve, a sensor, control circuitry, and/or other suitable elements. When the bladder 212 is inflated, the cuff 210 applies a pressure to, for example, the finger. The pressure applied to the finger by the cuff 210 may be the same as the pneumatic pressure in the bladder 212.

In an examples, the system 100 is configured to and comprises suitable means to pressurize the arm cuff 150 and to operate a volume clamp measurement on the finger cuff 104, Thus, the system is configured to and includes a means to change the pressure in the finger cuff 104 at a sufficient rate. Thus, the system 100 can include an inflation means to inflate the arm cuff 150, while in other alternatives, the determination of the measure indicative of mean systemic filling pressure also works when the arm cuff 150 is inflated by another device such as an oscillometric BP device, which is not part of the system 100.

In other examples of the arm cuff 150 of FIG. 1, it is not necessary to have an arterial pulsatility sensor 214 and it is sufficient to provide a pressure generating and regulating system 220 which is capable of applying a pressure to, for instance, above systolic pressure.

In one embodiment, the arterial pulsatility sensor 214 may comprise a plethysmograph. The plethysmograph may make continuous volumetric measurements (or plethysmogram) of arterial blood flows within the finger. Thus, pulsatility in the finger may be detected based on the plethysmogram. In one embodiment, the plethysmograph may comprise a light-emitting diode (LED)-photodiode pair. The LED may be used to illuminate the finger skin and light absorption, or reflection may be detected with the photodiode. Therefore, the plethysmogram may be generated based on the signal received from the photodiode.

The pressure generating and regulating system 220 and the arterial pulsatility sensor 214 may be connected to a control circuitry 230. The control circuitry 230 may instruct the pressure generating and regulating system 220 to inflate or deflate the bladder 212 based on a pressure setting, may receive pulsatility information from the pulsatility sensor 214, and may carry out necessary data manipulations.

Referring to FIG. 3, a block diagram illustrating example control device 230 is shown. The control device 230 may integrate parts of or all of the functions of the inflation control unit, the pressure signal monitoring unit, and the deriving unit of the system for a non-invasive blood pressure measurement according to the present disclosure. It should be appreciated that FIG. 3 illustrates a non-limiting example of a control device 230 implementation. Other implementations of the control device 230 not shown in FIG. 3 are also possible. The control device 230 may comprise a processor 310, a memory 320, and an input/output interface 330 connected with a bus 340. Under the control of the processor 310, data may be received from an external source through the input/output interface 330 and stored in the memory 320, and/or may be transmitted from the memory 320 to an external destination through the input/output interface 330. The processor 310 may process, add, remove, change, or otherwise manipulate data stored in the memory 320. Further, code may be stored in the memory 320. The code, when executed by the processor 310, may cause the processor 310 to perform operations relating to data manipulation and/or transmission and/or any other possible operations.

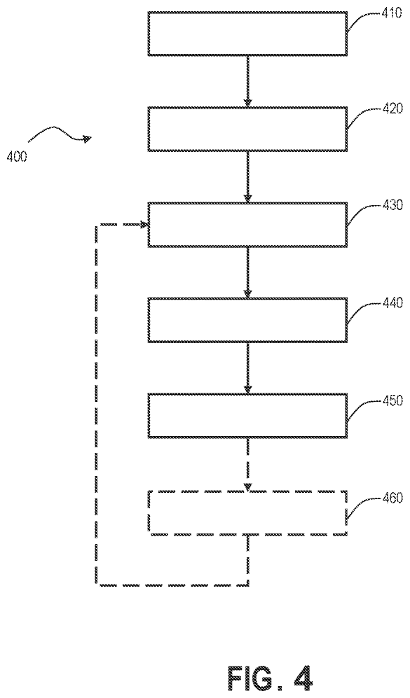

FIG. 4 schematically and exemplarily illustrates a method 400 for a non-invasive mean systemic filling pressure measurement.

Method 400 comprises a step 410 of applying a first pressure cuff such as arm cuff 150 to a limb of a living subject, in particular to an arm of a human being.

Next, in a step 420 a second pressure cuff such as finger cuff 104 is applied distally to the first pressure cuff to the same limb of the subject. Thus, the results of clamping the blood flow using the first pressure cuff can be determined at the position of the second pressure cuff.

In a step 430, the first pressure cuff is inflated to a pressure value exceeding a first threshold value. The first threshold value is preferentially systolic blood pressure, such that the blood flow to the distal portions of the limb is blocked.

In a step 440, the pressure signal, i.e., the blood pressure, of the second pressure cuff is monitored, for instance using the volume clamp method. The monitoring of step 440 preferentially starts with the first pressure cuff exceeding the first threshold value or some predetermined period after inflation of the first pressure cuff and proceeds for at least a certain, preferentially predefined duration. During the monitoring period, the pressure of the first pressure cuff is maintained above the first threshold value.

In some embodiments, the predefined duration of the monitoring period is a short monitoring period of at least 30s, wherein the measure indicative of MSFP is then determined preferentially based on a mathematical extrapolation, e.g. from an exponential decay function. In the alternative, the monitoring period can be extended to a long monitoring period, for instance to at least 60s, until the plateau phase is reached. In this case, no extrapolation is necessary and the asymptotic value can be directly determined as the reached asymptotic value. Also intermediate durations of the monitoring period are feasible which will increase the reliability and accuracy of the mathematical extrapolation.

In order to reduce the burden caused by the inflated first cuff on the patient, the extrapolation method involving a shorter monitoring period can be preferred. Nevertheless, it might be desirable to verify the reliability of the mathematical extrapolation by employing the longer monitoring period, i.e. the monitoring period which allows the plateau phase to be reached. For subsequent or periodic repetitions of the determination of the measure indicative of MSFP, the monitoring duration may be varied. For instance, while a certain share of the measurements can be conducted using the short monitoring period, also the long monitoring period can be employed from time to time.

Preferentially the system can assess the pressure decay in the second pressure cuff and if the pressure change is small enough, e.g. below a predefined threshold, or the level of the extrapolation sufficiently stable, the inflation of the first pressure cuff can be ended.

In a step 450, based on the monitored pressure signal of the second pressure cuff, a measure indicative of mean systemic filling pressure, MSFP, is derived. For instance, the measure indicative of MSFP is obtained as an asymptotic value of an exponential decay function fitted to the monitored blood pressure signal.

After termination of the monitoring step 440, the pressure applied to the first pressure cuff may be released in an optional step 460. Further optionally, the steps 430-460 may be repeated periodically during treatment of the subject, for instance one or several times per hour, in order to continuously monitor the subject's MSFP.

It should be appreciated that aspects of the invention previously described may be implemented in conjunction with the execution of instructions or code by processors, circuitry, controllers, control circuitry, etc. (e.g., processor 310 of FIG. 3). As an example, control circuitry may operate under the control of a program, algorithm, code, routine, or the execution of instructions to execute methods or processes (e.g., method 400 of FIG. 4) in accordance with embodiments of the invention previously described. For example, such a program may be implemented in firmware or software (e.g., stored in memory and/or other locations) and may be implemented by processors, control circuitry, and/or other circuitry, these terms being utilized interchangeably. Further, it should be appreciated that the terms processor, microprocessor, circuitry, control circuitry, circuit board, controller, microcontroller, etc., refer to any type of logic or circuitry capable of executing logic, commands, instructions, software, firmware, functionality, etc., which may be utilized to execute embodiments of the invention.

The various illustrative logical blocks, processors, modules, and circuitry described in connection with the embodiments disclosed herein may be implemented or performed with a general purpose processor, a specialized processor, circuitry, a microcontroller, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor may be a microprocessor or any conventional processor, controller, microcontroller, circuitry, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module/firmware executed by a processor, or any combination thereof. A software module may reside in RAM memory, flash memory, ROM memory, EPROM memory, EEPROM memory, registers, hard disk, a removable disk, a CD-ROM, non-transitory computer readable medium, or any other form of storage medium known in the art. An exemplary storage medium is coupled to the processor such the processor can read information from, and write information to, the storage medium. In the alternative, the storage medium may be integral to the processor.

Further examples of the invention:
Example 1. A method for a non-invasive determination of a physiological parameter, the method (400) comprising:
applying (410) a first pressure cuff (150) to a limb of a living subject, in particular of a human being,
applying (420) a second pressure cuff (104) distally to the first pressure cuff (150) to the limb of the subject,
inflating (430) the first pressure cuff (150) to a pressure value exceeding a first threshold value,
monitoring (440) a pressure signal of the second pressure cuff (104) after the step of inflating the first pressure cuff (150),
deriving (450) a measure indicative of mean systemic filling pressure, MSFP, from the monitored pressure signal of the second pressure cuff (104).
Example 2. The method (400) according to example 1, wherein the step of monitoring a pressure signal of the second pressure cuff (104) comprises a step of
determining a course of a decay of the pressure signal of the second pressure cuff (104).
Example 3. The method (400) according to example 2, wherein the step of deriving (450) a measure indicative of MSFP comprises the following step:
deriving an asymptotic value from the determined course of the decay as the measure indicative of MSFP.
Example 4. The method (400) according to example 3, wherein the step of deriving an asymptotic value comprises the following steps:
fitting the course of the decay of the pressure signal of the second pressure cuff to an exponential decay function, and
determining the asymptotic value from an extrapolation using the exponential decay function.
Example 5. The method (400) according to any of the preceding examples, further comprising
determining, in particular using the second pressure cuff (104), a systolic blood pressure of the subject, wherein the first threshold value is set equal to or above the systolic blood pressure.
Example 6. The method (400) according to any of the preceding examples, wherein the step of inflating (430) the first pressure cuff to a pressure value exceeding a first threshold value includes inflating the first pressure cuff using an inflation rate exceeding a second threshold value.
Example 7. The method (400) according to any of the preceding examples, wherein at least one of
a) the first pressure cuff (150) is an arm cuff applied to an arm of the living subject and
b) the second pressure cuff is a finger cuff (104) applied to a finger (105) of the arm of the living subject.
Example 8. The method (400) according to any of the preceding examples, wherein the steps of inflating (430) the first pressure cuff, monitoring (440) the pressure signal of the second pressure cuff and deriving (450) a measure indicative of MSFP are repeated, in particular periodically repeated.
Example 9. The method (400) according to any of the preceding examples, further comprising the following step:
assessing and compensating an influence of the position of the limb of the subject relative to a heart level of the subject using a hydrostatic pressure difference system.
Example 10. The method (400) according to example 9, further comprising the step of
assessing changes in the hydrostatic pressure difference system and using the assessed changes in a quality index, thereby assessing the possible reduced accuracy due to movement of the arm.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

1. A system (102) for a non-invasive determination of a physiological parameter, comprising:
a first pressure cuff (150) configured to be applied to a limb of a living subject, in particular of a human being,
a second pressure cuff (104) configured to be applied distally to the first pressure cuff to the limb of the subject,
a inflation control unit (220) configured to inflate the first pressure cuff (150) to a pressure value exceeding a first threshold value,
a pressure signal monitoring unit (120) configured to monitor a pressure signal of the second pressure cuff (104) after the inflation control unit (220) inflated the first pressure cuff (150),
a deriving unit (230) configured to derive a measure indicative of mean systemic filling pressure, MSFP, from the pressure signal of the second pressure cuff (104) monitored by the pressure signal monitoring unit (120).

2. The system (102) according to claim 1, wherein at least one of
a) the first pressure cuff (150) is an arm cuff configured to be applied to an arm of the living subject and
b) the second pressure cuff (104) is a finger cuff configured to be applied to a finger (105) of the arm of the living subject.

3. The system (102) according to claim 1, wherein at least one of the inflation control unit (220), the pressure signal monitoring unit (120) and the deriving unit (230) is integrated in a central processing device (310).

4. The system (102) according to claim 1, wherein
the pressure signal monitoring (120) unit is further configured to determine a course of decay of the pressure signal of the second pressure cuff.

5. The system (102) according to claim 4, wherein
the deriving unit (230) is further configured to derive a asymptotic value from the determined course of the decay as the measure indicative of the MSFP.

6. The system (102) according to claim 5, wherein
the deriving unit (230) is further configured to fit the course of decay of the pressure signal of the second pressure cuff to an exponential decay function, and to
determine the asymptotic value from an extrapolation using the exponential decay function.

7. The system (102) according to claim 1, wherein
the pressure signal monitoring (120) unit is further configured to determine, in particular using the second pressure cuff (104), a systolic blood pressure of the subject, wherein the first threshold value is set equal to or above the systolic blood pressure.

8. The system (102) according to claim 1, wherein
the inflation control unit (220) is further configured to inflate the first pressure cuff using an inflation rate exceeding a second threshold value.

9. The system (102) according to claim 1, wherein at least one of
a) the first pressure cuff (150) is an arm cuff applied to an arm of the living subject and
b) the second pressure cuff is a finger cuff (104) applied to a finger (105) of the arm of the living subject.

10. The system (102) according to claim 1, wherein the inflation control unit (220), the signal monitoring unit (120), and the deriving unit (230) are further configured to repeat the inflating, monitoring, and deriving, in particular, to repeat the inflating, monitoring, and deriving periodically.

11. The system (102) according to claim 1, wherein the system in further comprises a hydrostatic pressure difference system configured to assess and to compensate an influence of a position of the limb of the subject relative to a heart level of the subject.

12. The system according to claim 11, wherein the hydrostatic pressure difference system is further configured to assess changes in the hydrostatic pressure difference system and to use the assessed changes in a quality index, thereby assessing possible reduced accuracy due to movement of the limb.
